SEQUENCE LISTING (61 sequences)

SEQ ID NO: 1 - DNA, 28 bp, Artificial Sequence
  Description of Artificial Sequence Synthetic primer
aaactcgagggctgccactgattcttca

SEQ ID NO: 2 - DNA, 33 bp, Artificial Sequence
  Description of Artificial Sequence Synthetic primer
aaatctagactatttacagataaggcaaacacg

SEQ ID NO: 3 - DNA, 32 bp, Artificial Sequence
  Description of Artificial Sequence Synthetic primer
aaactcgagagtgtggtgtcgaatagtttgct

SEQ ID NO: 4 - DNA, 36 bp, Artificial Sequence
  Description of Artificial Sequence Synthetic primer
caatctagatactttatttttcaaacatcacatctc

SEQ ID NO: 5 - DNA, 33 bp, Artificial Sequence
  Description of Artificial Sequence Synthetic primer
aaagtcgacgtataaatagcctgtttgtatcga

SEQ ID NO: 6 - DNA, 27 bp, Artificial Sequence
  Description of Artificial Sequence Synthetic primer
aaatctagaacccgtgctatatgctca

SEQ ID NO: 7 - DNA, 41 bp, Artificial Sequence
  Description of Artificial Sequence Synthetic primer
aggaactcaactctaggtctttggatatgctccttgacccc

SEQ ID NO: 8 - DNA, 38 bp, Artificial Sequence
  Description of Artificial Sequence Synthetic primer
gagcatatccaaagacctagagttgagttcctttgggg

SEQ ID NO: 9 - DNA, 43 bp, Artificial Sequence
  Description of Artificial Sequence Synthetic primer
ccaaatccccactgaggtcttgaaaaacaaagtatttatgaat

SEQ ID NO: 10 - DNA, 35 bp, Artificial Sequence
  Description of Artificial Sequence Synthetic primer
actttgtttttcaagacctcagtggggatttgggg

SEQ ID NO: 11 - DNA, 41 bp, Artificial Sequence
  Description of Artificial Sequence Synthetic primer
tactagatacctcgaggtctttgtatagacttgtaaataga

SEQ ID NO: 12 - DNA, 40 bp, Artificial Sequence
  Description of Artificial Sequence Synthetic primer
acaagtctatacaaagacctcgaggtatctagtaagggtt

SEQ ID NO: 13 - DNA, 34 bp, Artificial Sequence
  Description of Artificial Sequence Synthetic oligonucleotide
ctagctcgagatacatacttctaatcattccata

SEQ ID NO: 14 - DNA, 34 bp, Artificial Sequence
  Description of Artificial Sequence Synthetic oligonucleotide
ctagtatggaatgattagaagtatgtatctcgag

SEQ ID NO: 15 - DNA, 30 bp, Artificial Sequence
  Description of Artificial Sequence Synthetic primer
aaactcgagccttgatttgacatgttttcc

SEQ ID NO: 16 - DNA, 27 bp, Artificial Sequence
  Description of Artificial Sequence Synthetic primer
aaactcgagggcacgagcatggctctg

SEQ ID NO: 17 - DNA, 30 bp, Artificial Sequence
  Description of Artificial Sequence Synthetic primer
aaactcgagcaccttgaaaagatgacttca

SEQ ID NO: 18 - DNA, 25 bp, Artificial Sequence
  Description of Artificial Sequence Synthetic oligonucleotide
aaagtgctagagttgagttcctttg

SEQ ID NO: 19 - DNA, 25 bp, Artificial Sequence
  Description of Artificial Sequence Synthetic oligonucleotide
ttcttaaatacatatttttggggtc

SEQ ID NO: 20 - DNA, 23 bp, Artificial Sequence
  Description of Artificial Sequence Synthetic oligonucleotide
atggaatgattagaagtatgtat

SEQ ID NO: 21 - DNA, 25 bp, Artificial Sequence
  Description of Artificial Sequence Synthetic oligonucleotide
taaatactttgtttttcaagtgctc

SEQ ID NO: 22 - DNA, 25 bp, Artificial Sequence
  Description of Artificial Sequence Synthetic oligonucleotide
ccatttctaaagcttacattacata

SEQ ID NO: 23 - DNA, 25 bp, Artificial Sequence
  Description of Artificial Sequence Synthetic oligonucleotide
aaagtgctcgaggtatctagtaagg

SEQ ID NO: 24 - DNA, 25 bp, Artificial Sequence
  Description of Artificial Sequence Synthetic oligonucleotide
atgtcaaatcaaggtaataatccac

SEQ ID NO: 25 - DNA, 23 bp, Artificial Sequence
  Description of Artificial Sequence Synthetic oligonucleotide
agctggatgaacagagccatgct

SEQ ID NO: 26 - RNA, 29 nt, Danio rerio
accccaaaggaacucaacucuagcacuuu

SEQ ID NO: 27 - RNA, 21 nt, Artificial Sequence
  Description of Artificial Sequence Synthetic oligonucleotide
aaagugcuaucaaguuggggu

SEQ ID NO: 28 - RNA, 29 nt, Danio rerio
accccaaaggaacucaacucuaggucuuu

SEQ ID NO: 29 - RNA, 27 nt, Danio rerio
acuccccaaauccccacugagcacuug

SEQ ID NO: 30 - RNA, 22 nt, Artificial Sequence
  Description of Artificial Sequence Synthetic oligonucleotide
uaagugcuucucuuugggguag

SEQ ID NO: 31 - RNA, 27 nt, Danio rerio
acuccccaaauccccacugaggucuug

SEQ ID NO: 32 - RNA, 43 nt, Danio rerio
acuccccaaauccccacugagcacuugaaaaacaaaguauuua

SEQ ID NO: 33 - RNA, 48 nt, Danio rerio
ccccaaaggaacucaacucuagcacuuuggauaugcuccuugacccca

SEQ ID NO: 34 - RNA, 48 nt, Tetraodon nigroviridis
cgcggggguucggacccgcaggcacuugauagauguuuucgguuugaa

SEQ ID NO: 35 - RNA, 48 nt, Takifugu rubripes
caggcacagaggagguucgaggcacuugacagauguuuucagucugag

SEQ ID NO: 36 - RNA, 48 nt, Oryzias latipes
ucaguggauuuauguacacaggcacuucacagagaggacaucccucac

SEQ ID NO: 37 - RNA, 48 nt, Gasterosteus aculeatus
aaaguggauggacguccacaggcacuuuauguccgguuugacacugac

SEQ ID NO: 38 - RNA, 48 nt, Xenopus tropicalis
cauuuccccuaauacagagcagcacuucuuguuccacuauuuauugua

SEQ ID NO: 39 - RNA, 48 nt, Xenopus laevis
aagaugaaggaaauauucaaggcacuucauaaaaauaaagacuguguu

SEQ ID NO: 40 - RNA, 48 nt, Xenopus laevis
ggauccacauuuuacagagcagcacuucuugucccacuauuuauugug

SEQ ID NO: 41 - RNA, 48 nt, Ciona savignyi
uuauagauuuucuuuuucuuugcacuuuucaagaaauauuaauuuuua

SEQ ID NO: 42 - RNA, 48 nt, Mus musculus
ugccuuugaccagagaggugggcacuuguccaagaggggacuggccau

SEQ ID NO: 43 - RNA, 48 nt, Danio rerio
gacuccccaaauccccacugagcacuugaaaaacaaaguauuuaugaa

SEQ ID NO: 44 - RNA, 48 nt, Danio rerio
aacccuuacuagauaccucgagcacuuuguauagauuguaaauagagc

SEQ ID NO: 45 - RNA, 48 nt, Takifugu rubripes
gccacgaaaccccccccaccagcacuuuuuuuguaacuauuuuguaaa

SEQ ID NO: 46 - RNA, 48 nt, Takifugu rubripes
acaaucauggucacucuauaagcacuuuuaauuaagauuugauuaaua

SEQ ID NO: 47 - RNA, 48 nt, Tetraodon nigroviridis
ccucagaaacuccccccaccagcacuuuuuuguaucuauuuuguacau

SEQ ID NO: 48 - RNA, 48 nt, Tetraodon nigroviridis
cagacgccagcgucucuagaagcacuuuuaauucaaauuaaauugaca

SEQ ID NO: 49 - RNA, 48 nt, Xenopus laevis
augauagaagcauuuagagaagcacuuaccuguaaauagacugauaua

SEQ ID NO: 50 - RNA, 48 nt, Xenopus laevis
gggcccagugcagcugccccugcacuuacccugaagacagcccuguua

SEQ ID NO: 51 - RNA, 48 nt, Mus musculus
guucacugcacuguaugcgaagcacuuacauguauagauacugcaaac

SEQ ID NO: 52 - RNA, 48 nt, Mus musculus
cuuguccaucacccaguuuaagcacuuacauggguaaaucaugucacu

SEQ ID NO: 53 - RNA, 48 nt, Homo sapiens
auucacugcacuauauucuaagcacuuacauguggagauacuguaacc

SEQ ID NO: 54 - RNA, 48 nt, Homo sapiens
cccguccaucacccauccuaagcacuuacgugaguaaauaaugcagcu

SEQ ID NO: 55 - RNA, 48 nt, Danio rerio
ugauguucggacccgaacgcagcacuugcugucucaucagcuucaugc

SEQ ID NO: 56 - RNA, 48 nt, Xenopus tropicalis
caaaaacaaaaagcgaaaacagcacuugaggucccaucaacguaaagc

SEQ ID NO: 57 - RNA, 48 nt, Takifugu rubripes
acagacauguuuggaaaauuggcacuuuguuacccauugauaugcacc

SEQ ID NO: 58 - RNA, 48 nt, Tetraodon nigroviridis
acagacauguuuagaaaauuggcacuuuguuacccauugauaugcacc

SEQ ID NO: 59 - RNA, 48 nt, Mus musculus
aaaaauuccagagcaaaaacagcacuugaggucucaucaguuaaagca

SEQ ID NO: 60 - RNA, 48 nt, Homo sapiens
aaaaguucaagagagaaaacagcacuugaggucucaucaauuaaagca

SEQ ID NO: 61 - RNA, 22 nt, Artificial Sequence
  Description of Artificial Sequence Synthetic oligonucleotide
uaagugcuauuuguugggguag

(19) United States
(12) Patent Application Publication
(10) Pub. No.: US 2011/0009466 A1
(43) Pub. Date: Jan. 13, 2011

(54) METHODS OF INCREASING GENE EXPRESSION THROUGH RNA PROTECTION

(75) Inventors: Alexander F. Schier, Cambridge, MA (US); Antonio J. Giraldez, Hamden, CT (US); Wen-Yee Choi, Durham, NC (US)

Correspondence Address: ROPES & GRAY LLP;IPRM - Floor 43, PRUDENTIAL TOWER, 800 BOYLSTON STREET, BOSTON, MA, 02199-3600, US

(73) Assignee: President and Fellows of Harvard College, Cambridge, MA (US)

(21) Appl. No.: 12/675,965

(22) PCT Filed: Aug. 22, 2008
(86) PCT No.: PCT/US08/09987

§ 371 Date: Sep. 24, 2010

Related U.S. Application Data
(60) Provisional application No. 60/966,867, filed on Aug. 29, 2007.

(51) Int. Cl. A61K 31/7088; C12N 5/00; A61P 43/00

(52) U.S. Cl. 514/44 A; 435/375

(57) ABSTRACT

This invention relates to the use of one or more RNA target protectors to inhibit the binding of an RNA, e.g., small RNA, to a target RNA (e.g., a target mRNA), thus increasing the stability of the target RNA and its function (e.g., increasing the gene expression of the gene corresponding to a target mRNA). An RNA target protector may be, for example, an oligonucleotide, e.g., a morpholino, or a small molecule. The invention further relates to the treatment of a human patient in need thereof with one or more RNA target protectors.

### REFERENCE TO RELATED APPLICATIONS

This application claims the benefit of the filing date under 35 U.S.C. §119(e) to U.S. Provisional Application No. 60/966,867, filed on Aug. 29, 2007, the entire content of which (including the specification and drawings) is incorporated herein by reference.

### STATEMENT REGARDING FEDERALLY SPONSORED RESEARCH

Work described herein was supported by Grant No. GM-56211 from the National Institutes of Health (NIH). The U.S. Government has certain rights in the invention.

### FIELD OF THE INVENTION

This invention relates to the use of one or more RNA target protectors to inhibit the interaction of an RNA, e.g., small RNA (miRNA, etc.), with a target RNA (e.g., mRNA), thus increasing the stability of the target RNA and its function (e.g., gene expression of the gene corresponding to a target mRNA). An RNA target protector of the invention may be, for example, an oligonucleotide, e.g., a morpholino, or a small molecule. The invention further relates to the treatment of a human patient in need thereof with one or more RNA target protectors of the invention.

### BACKGROUND OF THE INVENTION

miRNAs are RNAs of approximately 22 nucleotides in length that block the translation and enhance the decay of target mRNAs (Bushati and Cohen, Annu Rev Cell Dev Biol (2007)). Recent studies have uncovered functions of specific miRNA families and have identified hundreds of putative target mRNAs (Bushati and Cohen, Annu Rev Cell Dev Biol (2007); Kloosterman and Plasterk, Dev Cell 11:441-50 (2006); Rajewsky, Nat Genet 38 Supple:S8-13 (2006)).

siRNAs are exogenous double-stranded RNAs of approximately 20-25 nucleotides in length that can drastically reduce expression of a targeted gene via cleavage by the endonuclease Argonaute when the mRNA of the gene is complexed with the siRNA. There is considerable interest in utilizing siRNAs as therapeutic agents for a wide range of diseases including infectious diseases and different forms of cancer. One potential obstacle to the therapeutic use of siRNAs is that siRNAs may have affinity for mRNAs other than the intended target. The undesired reduction in expression of non-target mRNAs in response to siRNA treatment is known as the siRNA off-target effect.

Another class of small RNAs, the Piwi-interacting RNAs (piRNAs), was identified through association with Piwi proteins in mammalian testes (Aravin et al., 2006; Girard et al., 2006; Grivna et al., 2006; Lau et al., 2006). These RNAs range from 26-30 nucleotides in length and are produced from discrete loci. Generally, genomic regions spanning 50-100 kB in length give rise to abundant piRNAs with profound strand asymmetry. Although the piRNAs themselves are not necessarily conserved, even between closely related species, the positions of piRNA loci in related genomes are conserved, with virtually all major piRNA-producing loci having synthetic counterparts in mice, rats and humans (Girard et al., 2006). Interestingly, the loci and consequently the piRNAs themselves are relatively depleted of repeat and transposon sequences, with only 17% of human piRNAs corresponding to known repetitive elements as compared to a nearly 50% repeat content for the genome as a whole. Despite the apparent differences in the content of RNA populations associated with Piwi proteins in mammals and Drosophila, Piwi family proteins share essential roles in gametogenesis, with all three murine family members, Miwi2, Mili, and Miwi, being required for male fertility.

### BRIEF SUMMARY OF THE INVENTION

The invention relates to novel therapeutic compounds and methods for protecting target RNA, e.g., mRNA, against undesired interaction with other RNA molecules, e.g., small RNAs including miRNA, siRNA and piRNA. The present invention contemplates a method comprising delivering a target protector to a target RNA. The target protector interacts with its target RNA in a sequence specific manner, typically by binding to the target RNA, thereby interfering with the ability of the target RNA to interact, e.g., bind, to other RNA molecules. The binding of the target protector preferably does not substantially affect the function of the target RNA, at least not permanently. For example, when the target RNA is mRNA, binding of a target protector to the target mRNA prevents the mRNA from interacting with other RNA molecules (e.g., the inhibitory miRNA or siRNA) at the site where the target protector is bound. Thus, degradation or translational repression of the target mRNA is inhibited, and expression of the gene corresponding to the target mRNA can be increased relative to the expression of the same gene in the absence of the target protector.

Thus, one aspect of the invention provides a method of inhibiting the interaction between a target RNA (such as an mRNA) and a small RNA, comprising contacting the target RNA (e.g., mRNA) with a target protector that binds to the target RNA in a sequence specific manner and inhibits interaction between the small RNA and the target RNA.

In one embodiment, the small RNA may be an siRNA, a piRNA, or an miRNA.

The target RNA may be an mRNA, ncRNA, snRNA, rRNA, tRNA, snoRNA, XIST RNA, or other RNA. It is specifically noted that although many of the embodiments described herein are exemplified with respect to mRNA target molecules and miRNAs, the invention is clearly not intended to be limited to such embodiments. Such illustrative embodiments are used for ease of description only, and other RNA targets and RNA molecules may be substituted for mRNA and miRNA, respectively, as described above and throughout.

In one embodiment, the present invention provides a method of inhibiting the interaction between mRNA and a small RNA in vivo, comprising administering to an organism an effective amount of an mRNA target protector, wherein the mRNA target protector binds to the mRNA in a sequence specific manner and inhibits interaction between the small RNA and the mRNA.

In one embodiment, the mRNA target protector is an oligonucleotide, such as an oligonucleotide at least 75% complementary to the target RNA (mRNA), preferably at least 80% complementary, and even more preferably at least 90% complementary to the target RNA (mRNA). In another embodiment, the oligonucleotide is at least 95% complementary to the target RNA (mRNA), more preferably at least 97% complementary to the target RNA (mRNA), and even more preferably at least 99% complementary to the target RNA (mRNA). In another embodiment, the oligonucleotide is 100% complementary to the target RNA (mRNA).

In another embodiment, the mRNA target protector has one or more modifications, such as 3′ end modifications and 5′ end modifications. In another embodiment, the mRNA target protector is an antisense oligonucleotide. In another embodiment, the mRNA target protector is an RNase H-independent oligonucleotide. In another embodiment, the mRNA target protector is an RNase H-competent oligonucleotide. In another embodiment, the mRNA target protector is a morpholino, PNA, or other polynucleotide analogs. In another embodiment, the mRNA target protector is a small molecule, such as one that specifically binds to the target sequence on the target RNA.

In certain embodiments, the target protector binds to a target region in the 3′-UTR of the target RNA (mRNA). In certain embodiments, the target protector may bind to a target region of the target RNA, the target region being complementary to the miRNA seed region. The target protector may further bind to a 3′-UTR flanking sequence of the target region. For example, one end of the target protector may bind the flanking sequence, and the other end of the target protector may bind the target region, or vice versa.

In certain embodiments, the small RNA may be endogenous or exogenous.

In certain embodiments, contacting the target mRNA with the target protector results in an increase in gene expression of a gene encoding the mRNA. This can be useful for a number of therapeutic indications. For example, the gene may have a deficient expression level, and the target protector at least partially relieves the inhibitory effect of miRNA/siRNA on the target gene, leading to increased gene expression. Alternatively, the gene may have a relatively normal expression level, and the target protector may increase the normal expression level to achieve a beneficial effect.

In certain embodiments, the target protector does not substantially inhibit the expression from the target mRNA. By binding directly to the target RNA, the target protector does not significantly decrease the stability of the RNA, or blocks translation from a target mRNA.

In certain embodiments, the stability of the mRNA is increased in the presence of the mRNA target protector compared to that in the absence of the mRNA target protector.

In certain embodiments, translation from the mRNA is increased in the presence of the mRNA target protector compared to that in the absence of the mRNA target protector.

A related aspect of the invention provides an in vivo method of the invention, comprising administering an effective amount of the mRNA target protector to an organism expressing the mRNA and the small RNA.

In one embodiment, the mRNA target protector is administered to an organism selected from the group consisting of human, primate, mouse, rat, cow, pig, horse, goat, dog, cat, frog, zebrafish, fly, worm, and plant. In another embodiment, the organism is a human patient in need of treatment for a disease characterized by a deficiency in expression from the mRNA. In another embodiment, the mRNA target protector is delivered using a delivery method selected from the group of oral delivery, intravenous delivery, inhalation, percutaneous delivery, vaginal delivery, and rectal delivery.

In certain embodiments, the method of the invention is carried out in vitro, or in a cell, such as a cultured cell, including a cell cultured outside of a living organism. The cell may be from an organism selected from human, primate, mouse, rat, cow, pig, horse, goat, dog, cat, frog, zebrafish, fly, worm, or plant.

In one embodiment, administering the mRNA target protector to the organism results in an increase in the gene expression level of the gene corresponding to the mRNA. In another embodiment, the gene is a gene with a deficient expression level in the organism. In another embodiment, the gene is a gene with a normal expression level in the organism.

In another embodiment, the invention provides a method of inhibiting the interaction between mRNA and a small RNA, comprising contacting a cell with an effective amount of an mRNA target protector, wherein the mRNA target protector binds to the mRNA in a sequence specific manner and inhibits interaction between the small RNA and the mRNA, whereby the gene expression level of the gene corresponding to the mRNA is increased relative to the expression level of the same gene in the absence of the target protector. In one embodiment the cell is cultured outside of a living organism.

In one embodiment, the invention provides a method of inhibiting the interaction between mRNA and a small RNA in vitro, comprising contacting an mRNA with an effective amount of an mRNA target protector, wherein the mRNA target protector binds to the mRNA in a sequence specific manner and inhibits interaction between the small RNA and the mRNA, whereby the level of the mRNA is increased relative to the level of the mRNA in the absence of the target protector. In another embodiment, the invention provides a method of inhibiting the interaction between mRNA and a small RNA in vitro, comprising contacting an mRNA with an effective amount of an mRNA target protector, wherein the mRNA target protector binds to the mRNA in a sequence specific manner and inhibits interaction between the small RNA and the mRNA, whereby the translation of the mRNA is increased relative to the translation of the mRNA in the absence of the target protector.

The invention described herein has many uses. For example, target protectors enable the study of the physiological function of specific miRNA-mRNA target pairs that cannot be uncovered in miRNA knockouts. In addition, there are multiple potential therapeutic applications of target protectors. For example, assume an miRNA normally regulates mRNA X; where decreased production of gene product as a result of haplo-insufficiency, regulatory mutation, or hypomorphic allele results in disease, delivery of an mRNA target protector can increase production of the gene product to improve or relieve the disease. Furthermore, assume mRNA X is not normally regulated by a miRNA, and misregulation of mRNA X by a miRNA leads to disease; delivery of an mRNA target protector can prevent this misregulation. Analogously, target mRNAs can be protected from overexpression of a miRNA that normally regulates mRNA X. In addition mRNA target protectors can be used to increase the levels of a beneficial gene product X in the wild-type context by inhibiting miRNA-induced repression.

Analogously, the subject target protectors can be used to protect target RNAs from other small RNAs, such as siRNAs or piRNAs.

It should be understood that the various embodiments described herein can be combined with one another wherever applicable, including the embodiments described under different aspects of the invention.

### BRIEF DESCRIPTION OF THE DRAWINGS

FIGS. 1A-1F show that miR-430 represses sqt and lft2 expression and activity. (A) mRNAs for GFP reporters (green) containing the 3′ UTRs of sqt or lft2 are co-injected with control DsRed (red) mRNA. Expression is analyzed at 25-30 hours post-fertilization (hpf). Wild-type (WT) reporters are repressed in wild-type embryos compared to MZdicer mutants. Repression is abolished by mutations in the predicted miR-430 target sites. Predicted pairings of the 3′UTRs to miR-430 are shown. The lft2 reporter appears more strongly repressed by miR-430 than the sqt reporter. (B) Outline of activity assays; sqt or lft2 open reading frame (ORF) with either wild-type or mutated 3′UTR is injected at the one cell stage. mRNA activity is assessed at 50% epiboly (˜5 hpf) by RNA in situ hybridization or at 25-30 hpf by morphology. (C) Embryos injected with 2 pg of wild-type lft2 mRNA appear similar to uninjected controls whereas 2 pg lft2mut-3′UTR mRNA causes cyclopia (arrow) and loss of trunk mesoderm (arrowhead), hallmarks of reduced Nodal signaling. (D) Physiological activity of sqt or lft2 mRNA assessed by fascin (fas) induction, a marker for Nodal signaling activity. 2 pg of lft2mut-3′UTR mRNA causes a stronger decrease in fas induction than wild-type lft2. 2 pg of sqtmut-3′UTR mRNA leads to greater ectopic induction of fas than wild-type sqt. (E) Percentage of embryos with decreased Nodal signaling (cyclopia and loss of trunk mesoderm) at increasing concentrations of wild-type lft2 or lft2mut-3′UTR mRNA. (F) Percentage of embryos with increased Nodal signaling (ectopic gsc induction covering >50% of the animal pole) at increasing concentrations of wild-type sqt or sqtmut-3′UTR mRNA.

FIGS. 2A-2I show that miRNA target protectors (TPs) interfere with specific miRNA-mRNA interactions. (A) Experimental approach. Target protectors are co-injected with GFP-reporters (green) into wild-type embryos and prevent miR-430-induced target repression. Predicted pairings of sqt-TP^{miR-430} and lft2-TP^{miR-430} to sqt and lft2 3′UTRs are shown. DsRed mRNA (red) is injected as control. (B) Wild-type reporter is repressed in wild-type embryos. (C and D) Co-injection of sqt-TP^{miR-430} or mutation of miR-430 target site prevents GFP repression. (E) sqt-TP^{miR-430} does not affect repression of lft2-GFP reporter. (F) sqt-GFP reporter with introduced miR-1 target site is repressed in wild-type embryos. Note that miR-1 is not expressed during early zebrafish embryogenesis. (G) sqt-TP^{miR-430} prevents GFP repression in the absence of miR-1. (H) sqt-TP^{miR-430} does not interfere with activity of injected miR-1. (I) sqt-TP^{mir-1} does not interfere with miR-430 activity.

FIGS. 3A-3D show that target protection results in increased sqt and lft2 expression and activity. (A) sqt-TP^{miR-430} injection results in elevated sqt expression, similar to MZdicer mutants. sqt-TP^{miR-430} does not increase sqt expression in MZdicer. (B) lft2-TP^{miR-430} injection results in elevated lft2 expression, similar to MZdicer mutants. lft2-TP^{miR-430} does not increase lft2 expression in MZdicer. (C) sqt-TP^{miR-430}-injected embryos exhibit increased gsc expression (arrowheads) that is suppressed by co-injection of a sqt-AUG morpholino. (D) lft2-TP^{miR-430}-injected embryos display cyclopia (arrowheads) that is suppressed by co-injection of a lft2-AUG morpholino.

FIGS. 4A-4F show that miR-430 maintains the balance between sqt and lft2. (A) Schematics of miR-430 regulation of sqt (S) and lft2 (L) in wild type, wild type+sqt-TP^{miR-430}, wild type+lft2-TP^{miR-430}, wild type+sqt-TP^{miR-430}+lft2-TP^{miR-430}, MZdicer, and Mzdicer miR-430 embryos. Removal of miR-430 regulation in each case results in increased sqt and/or lft2 expression. (B) gsc expression is increased in sqt-TP^{miR-430}-injected embryos and decreased in lft2-TP^{miR-430}-injected embryos. gsc induction is similar in wild type, wild type+sqt-TP^{miR-430}+lft2-TP^{miR-430}, MZdicer and MZdicer miR-430 embryos at 50% epiboly. (C) sox17 expression is reduced in wild type+sqt-TP^{miR-430}+lft2-TP^{miR-430} and MZdicer embryos compared to uninjected wild types at 75% epiboly. sox17 labels endodermal cells (bracket) and forerunner cells (arrowhead). (D) Quantification of sox17-expressing endodermal cells (n=5-10 embryos for each genotype/injection). (E) Quantification of sox17-expressing forerunner cells (n=12-35 embryos for each genotype/injection). (D and E) Endodermal and forerunner cell numbers vary from embryo to embryo. Bars represent mean±SEM, which are significantly different between wild type and wild type+sqt-TP^{miR-430} (p<0.0005 by two-tailed Student's t test), wild type and wild type+lft2-TP^{miR-430} (p<10-12), wild type and wild type+sqt-TP^{miR-430}+lft2-TP^{miR-430} (p<10-7), wild type and MZdicer (p<10-8), wild type+lft2-TP^{miR-430} and wild type+sqt-TP^{miR-430}+lft2-TP^{miR-430} (p<0.02), MZdicer and Mzdicer miR-430 (p<10-5) embryos. (F) Model for miRNA-mediated balancing of an agonist and an antagonist.

FIGS. 5A-5C demonstrate that Nodal signaling components are targeted by miR-430. (A) Nodal signaling pathway. The Nodals Squint and Cyclops are antagonized by Lefty1 and Lefty2. Nodal signaling activates target genes including gsc, fas, and sox17. The agonist Squint (green), antagonists Lefty1 and Lefty2 (red), and Smad2 (orange) contain putative miR-430 target sites. (B) Schematic of expression patterns of Nodal target genes at 50% epiboly. sqt, lft, and fas are expressed all around the margin while gsc is expressed only at the dorsal margin (animal view). (C) Conservation of miR-430 target sites in squint/nodal, lefty and smad2.

FIGS. 6A-6F show regulation of lft1 by miR-430. (A) The lft2 3′UTR is more strongly repressed by miR-430 than the lft1 3′UTR as shown by comparison of GFP reporters with wild-type or mutated 3′UTRs. (B) The lft2 3′UTR is more strongly repressed by miR-430 than the lft1 3′UTR as shown by comparison of GFP reporters in wild-type or MZdicer mutant embryos. (C) lft1-TP^{miR-430} prevents repression of lft1-GFP reporter. Repression of lft1-GFP reporter is not affected by lft2-TP^{miR-430}. (D) Wild-type embryos injected with increasing amounts of wild-type lft1 or lft1mut-3′ UTR mRNA. (E) Percentage of embryos with reduced Nodal signaling (cyclopia and loss of trunk mesoderm) at increasing concentrations of wild-type lft1 or lft1mut-3′UTR mRNA. (F) lft1 expression in wild-type and MZdicer embryos at 50% epiboly.

FIG. 7 shows concentration-dependence of sqt-TP^{miR-430} and lft2-TP^{miR-430}. sqt-TP^{miR-430} and lft2-TP^{miR-430} prevent GFP repression in wild-type embryos.

FIG. 8 shows binding to 3′UTR is not sufficient for protection. Sqt-TP^{control} is a morpholino designed to bind the sqt 3′UTR downstream of sqt-TP^{miR-430}. Sqt-TP^{control} does not affect sqt-GFP reporter repression.

FIG. 9 shows the specificity of TP^{miR-430}. Sqt-TP^{miR-430}, lft2-TP^{miR-430}, and lft1-TP^{miR-430} prevent GFP repression in wild-type embryos (left) but do not further increase GFP expression in MZdicer embryos (right). Mutation of the miR-430 target site does not affect GFP expression in MZdicer embryos.

FIG. 10 shows that sqt-TP^{miR-430} and lft2-TP^{miR-430} modulate Nodal signaling. Extended version of FIGS. 3C and 3D and 4B. (A-H) gsc expression domain marks dorsal mesoderm at the onset of gastrulation. (A′ and B′) Lateral view of embryo at onset of gastrulation. (A″ and B″) Embryonic shield marks dorsal mesoderm (outlined) at 6 hpf. (B, B′ and B″) Ectopic gsc induction corresponds to expanded shield (outlined). (C) sqt-AUG morpholino suppresses the ectopic gsc induction phenotype caused by sqt-TP^{miR-430}. (D) gsc induction is reduced by sqt-AUG morpholino. (E′) Frontal view of a 30 hpf wild type embryo; arrowheads indicate the distance between the retinas. (F and F′) Reduced gsc induction and cyclopia caused by lft2-TP^{miR-430}. (G and G′) Reduced gsc induction and cyclopia caused by lft2-TP^{miR-430} are suppressed by lft2-AUG morpholino. (H and H′) gsc induction and embryonic phenotype in lft2-AUG MO injected embryos.

FIGS. 11A-11C show the effects of sqt-TP^{miR-430} are caused by protection of zygotic sqt. (A) gsc induction is increased in sqt-TP^{miR-430}-injected wild-type embryos compared to sqt-TP^{control}-injected wild-type embryos (onset of gastrulation 5-6 hpf). (B and C) Embryonic mRNAs are provided both maternally and zygotically, i.e., by both the mother and the embryo (Tadros and Lipshitz, Dev Dyn 232: 593-608 (2005); A. F. Schier, Science 316: 406-7 (2007)). MiR-430 represses a large number of maternal mRNAs (A. J. Giraldez et al., Science 312: 75-9 (2006)). Since lft1 and lft2 are only expressed zygotically, our results show that miR-430 can also target zygotically expressed genes. Sqt mRNA is provided both maternally and zygotically (A. F. Schier, Annu Rev Cell Dev Biol 19: 589-621 (2003); Schier and Talbot, Annu Rev Genet 39: 561-613 (2005); B. Feldman et al., Nature 395: 181-5 (1998); Rebagliati et al., Dev Biol 199: 261-72 (1998)). To determine whether the effect of sqt-TP^{miR-430} was caused by its effects on maternal or zygotic sqt mRNA, we injected sqt-TP^{miR-430} into embryos that contain only maternal sqt mRNA (zygotic sqt mutants, Zsqt) or only zygotic sqt mRNA (maternal sqt mutants, Msqt) (B. Feldman et al., Nature 395: 181-5 (1998); T. O. Aoki et al., Dev Biol 241: 273-88 (2002)). (B) Expansion of gsc induction and higher levels of sqt in sqt-TP^{miR-430}-injected Msqt embryos. (C) Levels of gsc induction and sqt expression are unaffected in sqt-TP^{miR-430}-injected Zsqt embryos compared to sqt-TP^{control}-injected embryos.

FIGS. 12A-12D show some target protector functions and applications. (A) Target protectors enable the study of the physiological function of specific miRNA-mRNA target pairs that cannot be uncovered in miRNA knockouts. (B-D) Potential therapeutic applications of target protectors. (B) miRNA normally regulates mRNA X. Decreased production of gene product as a result of haplo-insufficiency, regulatory mutation, or hypomorphic allele results in disease. Target protection can increase production of gene product to relieve the diseased state: (C) mRNA X is not normally regulated by a miRNA. Misregulation of mRNA X by a miRNA leads to disease. Target protection can prevent this misregulation. Analogously, target mRNAs can be protected from overexpression of a miRNA that normally regulates X. (D) Target protectors may be used to increase the levels of a beneficial gene product X in the wild-type context by blocking miRNA-induced repression.

### DETAILED DESCRIPTION OF THE INVENTION

The invention relates to novel therapeutic compounds and methods for protecting target RNA, e.g., mRNA, against undesired interaction with other RNA molecules, e.g., small RNAs including miRNA, siRNA and piRNA. The present invention contemplates a method comprising delivering a target protector to a target RNA. The target protector interacts with its target RNA in a sequence specific manner, typically by binding to the target RNA, thereby interfering with the ability of the target RNA to interact, e.g., bind, to other RNA molecules. For example, when the target RNA is mRNA, binding of a target protector to the target mRNA prevents the mRNA from interacting with other RNA molecules (e.g., miRNA or siRNA) at the site where the target protector is bound. Thus, degradation and/or translational repression of the target mRNA is inhibited and expression of the gene corresponding to the target mRNA can be increased relative to the expression of the same gene in the absence of the target protector.

### DEFINITIONS

“In vivo” is used herein to refer to occurrence within a living whole organism.

“In vitro” is used herein to refer to occurrence outside a living cell. “In vitro” may refer to occurrence in cell extract or in a purified system.

“Small RNA” is used herein to refer to the class of endogenous and exogenous RNAs of lengths less than or equal to 100, 80, 60, 40, 30, 25 or 20 nucleotides, including, but not limited to, miRNAs, siRNAs, piRNAs, vmiRNAs, vault RNAs, Y RNAs, and rasi-RNAs. It will be understood through this disclosure that a value of “less than X” is intended to specifically disclose all incremental values less than that number. For example, “less than 100” is intended to specifically disclose 99, 98, 97, and so on down to and including 1.

“miRNA” is used herein to refer to an endogenous single-stranded RNA of approximately 22 nucleotides that decreases expression of one or more genes by base-pairing with the mRNA of the gene(s) and causing degradation of the target mRNA or blocking translation of the target mRNA.

“siRNA” is used herein to refer to an exogenous double-stranded RNA of approximately 20-25 nucleotides that decreases expression of one or more genes by base-pairing with the mRNA of the gene(s) and causing degradation of the target mRNA.

The terms “target protector,” and “TP” are used interchangeably herein to refer to any agent that inhibits the interaction between a target RNA and another RNA molecule that affects the expression of the gene corresponding to the mRNA. A target protector may comprise, for example, an oligonucleotide, an antisense oligonucleotide, an RNase H-independent oligonucleotide, an RNase H-competent oligonucleotide, a morpholino, or a small molecule. An oligonucleotide target protector may comprise DNA or RNA. An oligonucleotide target protector may additionally comprise a modified oligonucleotide such as, for example, a 2′O-Methyl oligonucleotide, a Peptide Nucleic Acid (PNA) oligonucleotide, or any other oligonucleotide that provides Watson-Crick base pairing with natural or modified nucleotides.

The term “RNA target” refers to any RNA to which a target protector binds in a sequence specific manner. Preferably the RNA target is one which has the ability to interact with another RNA molecule, wherein the interaction can be inhibited by a target protector.

The term “miRNA target site” refers to a region of mRNA that binds a miRNA.

“Morpholino” is used herein to refer to an oligonucleotide comprising standard nucleic acid bases bound to 6-membered morpholine rings which are connected by phosphorodiamidate groups.

“Sequence specific manner” is used herein to refer to a manner of binding in which a target protector binds to a target RNA sequence with greater affinity than the target protector binds to a different RNA sequence. An oligonucleotide, an antisense oligonucleotide, an RNase H-independent oligonucleotide, an RNase H-competent oligonucleotide, a morpholino, or a small molecule, may bind to a target RNA sequence in a sequence specific manner. An oligonucleotide or oligonucleotide variant that is at least 25%, 50%, 75%, 80%, 90%, 95%, 97%, 99%, or 100% complementary to its target RNA, e.g., mRNA, may bind in a sequence specific manner. As used herein it will be understood that a percentage value of “at least X %” is intended to specifically disclose all incremental values above and including the value. For example, “at least 50%” is intended to specifically disclose 50%, 51%, 52%, 53% and so on, up to and including 100%.

“Inhibiting the interaction” is used herein to refer to reducing the ability to interact and/or the duration of interaction between two molecules, e.g., between an mRNA and a miRNA. Such reduction may be partial or complete. In one embodiment the nature of the interaction is binding, e.g., Watson-Crick base pairing.

“Deficient expression level” is used herein to refer to a level of gene expression that is lower than an expression level of the same gene in a healthy or normal organism.

“Normal expression level” is used herein to refer to a level of gene expression that is the same as an expression level of the same gene in a healthy or normal organism.

### General Techniques

The practice of the present invention will employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), and Beaucage et al. eds., Curr. Protocols in Nucleic Acid Chemistry John Wiley & Sons, Inc., New York, 2000).

A therapeutic mRNA target protector may be designed and produced as follows. First, one or more therapeutically useful mRNA(s), relevant to a disease or condition, will be readily ascertainable by the skilled artisan, for example based on known and/or published studies. Potential miRNA binding sites can be identified in the mRNAs using methods known in the art, as described below. mRNA targets can be experimentally validated by applying an mRNA target protector to an organism and assaying for the expression level of a target gene, as disclosed herein. The present invention also contemplates that mRNA targets can be validated by applying an mRNA target protector to cultured cells and assaying the expression level/activity of a target gene. An experimentally validated mRNA target protector can be manufactured and formulated as a pharmacological or pharmaceutical preparation using methods known in the art and described below.

Selecting Target mRNAs

The target protector approach may be used to target a large number of genes relevant to human health, animal health, or agriculture. The present invention contemplates use of mRNA target protectors to raise the expression level of a gene or genes, wherein the expression is reduced by a disease or condition. The present invention also contemplates use of mRNA target protectors to raise the expression level of a gene or genes, wherein the expression level is normal, and increased expression would alleviate or slow the progression of a disease or condition. Potentially therapeutically relevant genes include insulin for the treatment of diabetes, dystrophin/dmd for the treatment of muscular dystrophy, p53 for the treatment of cancer, cftr for the treatment of cystic fibrosis, Rb for the treatment of cancer including retioblastoma, cyp21A2 for the treatment of Congenital adrenal hyperplasia, Brca1 for the treatment of cancer including breast cancer, and hcrt for the treatment of narcolepsy.

miRNAs are clearly relevant to human health and disease, as a mutation in the 3′ UTR of the gene slit/track improves its target site for miR189 and is linked to Tourette's syndrome. (Abelson J F et al, “Sequence variants in SLITRK1 are associated with Tourette's syndrome” Science. 2005 Oct. 14; 310(5746):317-20).

Predicting Small RNA Target Sites within a Selected Target RNA

Many predicted small RNA target sites have been identified, and methods of predicting miRNA target sites are known in the art. It has been estimated that more than 25% of all human genes are regulated by miRNAs. Since miRNA target sites often reside in the 3′ UTR of messages and 3′ UTR regions are often poorly conserved, cross-species comparisons of 3′ UTR regions can identify putative miRNA binding sites. Computational comparisons between conserved 3′ UTR regions and 7-nt “seed” sequences within miRNAs revealed multiple putative miRNA target sites (Lewis BP et al, “Prediction of Mammalian MicroRNA Targets” Cell, 115:787-798 (2003)). In addition, demanding that a conserved putative miRNA binding site correspond to a known or predicted miRNA sequence improved the signal to noise ratio for miRNA target site identification (Lewis BP et al, “Prediction of Mammalian MicroRNA Targets” Cell, 115:787-798 (2003)). Furthermore, the signal to noise ratio has been lowered further by demanding that a miRNA target site is present in multiple copies in the same 3′ UTR and by reducing the seed sequence to 6 nt (Lewis BP et al, “Conserved Seed Pairing, Often Flanked by Adenosines, Indicates that Thousands of Human Genes are MicroRNA Targets “Cell, 120:15-20 (2005)). In vivo, seed sequences are not the sole determinant of binding. Grimson et al used experimental and in silico techniques to identify characteristics of miRNA target sites that include AU-rich regions near the site, proximity of the miRNA target site to sites miRNAs that are expressed in similar tissues, the location of mRNA nucleotides pairing with miRNA nucleotides 13-16 near the miRNA target site seed sequence, location of the miRNA target site at least 15 nucleotides away from the stop codon, and location of the miRNA target site that is distant from the center of long 3′ UTRs (Grimson A et al, “MicroRNA Targeting Specificity in Mammals: Determinants beyond Seed Pairing” Molecular Cell, 27:91-105 (2007)). Since this site prediction model does not utilize cross-species sequence comparisons, it has identified poorly conserved or nonconserved miRNA sites. This site prediction model has also been used to identify siRNA off-target binding sites.

Designing mRNA Target Protectors

An mRNA target protector may be designed using methods described herein. Oligonucleotide mRNA target protectors, including morpholino mRNA target protectors, may be designed as perfectly complementary sequences to a miRNA target site. Methods for designing perfectly complementary nucleotide sequences are well known in the art (see, for example, Molecular Biology of the Cell, Fourth Edition, by Bruce Alberts, Alexander Johnson, Julian Lewis, Martin Raff, Keith Roberts, and Peter Walter; Garland; (March 2002)). In addition, an oligonucleotide mRNA target protector may be designed with modifications to the basic nucleic acid structure, such as, for example, 2′-O-methyl modifications. In addition, a given mRNA target protector may be designed with standard nucleotides and an alternative backbone such as, for example, a PNA backbone or a morpholino backbone.

mRNA target protectors used in the present invention were 25-nucleotide long morpholinos designed to bind to the region of the target mRNA complementary to the miRNA seed region (e.g., 6-8 nt miRNA target site) and to flanking sequences in the 3′UTR. One way to optimize the specificity of mRNA target protectors is to design the 3′ end of the mRNA target protector to bind to the target site while the 5′ region of the mRNA target protector binds to the unique downstream flanking sequences in the 3′UTR. Alternatively, mRNA target protectors can be designed to have their 5′ end bind to the target site while the 3′ region of the mRNA target protectors binds the upstream flanking sequences in the 3′UTR. Sequence specificity should be confirmed using whole genome sequence alignment programs such as BLAST. All morpholinos and mRNA target protector oligonucleotides were obtained from Gene Tools, but can be prepared by any vender.

Small molecule mRNA target protectors may be designed using methods known in the art. For example, one skilled in the art could readily utilize an assay for measuring the expression level of an mRNA target of interest. This assay could be, for example, reverse-transcriptase quantitative PCR, expression of a reporter gene such as GFP or luciferase, Northern blot, Western blot, or ELISA. Furthermore, one or more chemical libraries can be screened for the ability of particular molecules to elevate the expression level of the mRNA target of interest, and identified compounds can be tested for the ability to reduce binding of a relevant, e.g., miRNA to the mRNA target. Alternatively particular candidate molecules can be screened for the ability to reduce binding of a selected, e.g., miRNA, to an mRNA target without prior assessment of the candidate molecule's effect on expression level. Alternatively one skilled in the art could readily utilize an assay to measuring the binding of a small RNA to a target mRNA, such as by using Molecular Beacons fluorescent labels (http://www.molecular-beacons.org/) on synthetic small RNAs. One would then screen one or more chemical libraries to identify chemicals that alter the binding of the small RNA to the target RNA.

Producing mRNA Target Protectors

mRNA target protectors can be produced using methods known in the art. For example, DNA oligonucleotide target protectors can be produced chemically using methods known in the art (see, for example, U.S. Pat. No. 5,571,902). In addition, morpholino oligonucleotide mRNA target protectors can be produced chemically using methods known in the art (see, e.g., U.S. Pat. No. 5,602,240). In addition, small molecule mRNA target protectors oligonucleotide target protectors can be produced chemically using methods known in the art.

Administering mRNA Target Protectors to an Organism

An mRNA target protector can be formulated as a pharmacological preparation and administered to an organism using methods known in the art and techniques disclosed in the Example.

An mRNA target protector can be formulated as a pharmacological acceptable preparation. In certain embodiments, a pharmacological acceptable preparation includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

An mRNA target protector can be formulated with a pharmaceutically acceptable carrier including, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A health care professional may administer to a patient an effective amount of an mRNA target protector. The desired concentration of active compound delivered to the patient will depend on absorption, inactivation, and excretion rates of the drug as well as the delivery rate of the compound from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Further, the amounts of bioactive substances will vary depending upon the relative potency of the agents selected. Additionally, the optimal concentration and/or quantities or amounts of any particular therapeutic agent may be adjusted to accommodate variations in the treatment parameters. Such treatment parameters include the polymer composition of a particular preparation, the identity of the therapeutic agent utilized, and the clinical use to which the preparation is put, e.g., the site treated, the type of patient, e.g., human or non-human, adult or child, and the nature of the disease or condition.

In certain embodiments, subject compositions of the present invention maybe lyophilized or subjected to another appropriate drying technique such as spray drying.

Methods of preparing these formulations or compositions include the step of bringing into association subject compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated particles. An exemplary formulation for vaginal administration may comprise a bioactive agent that is a contraceptive or an anti-viral, anti-fungal or antibiotic agent.

Formulations which are suitable for vaginal administration also include pessaries, tampons, creams, gels, pastes, foams, or spray formulations containing such carriers as are known in the art to be appropriate.

Dosage forms for transdermal administration include powders, sprays, ointments, pastes, creams, lotions, gels, solutions, patches, and inhalants. A subject composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to subject compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a subject composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

The invention will be further described with reference to the following non-limiting example. References cited throughout this application are incorporated by reference in their entirety for all purposes.

### EXAMPLES

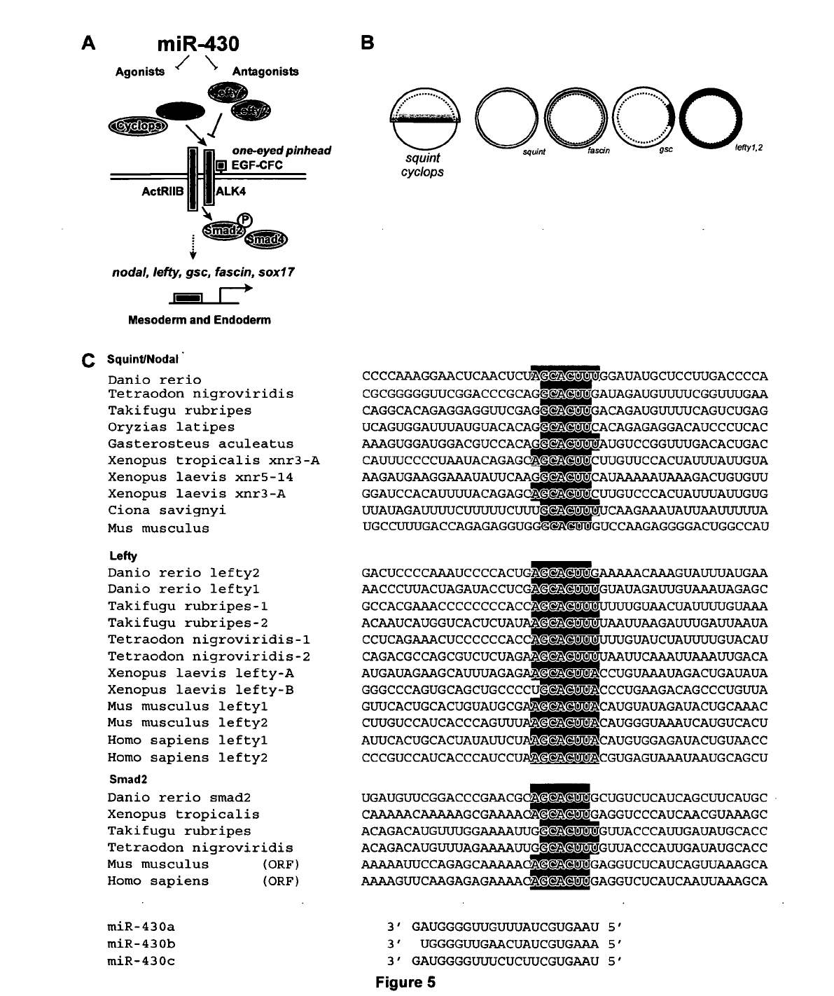

The specific examples provided herein focus on the zebrafish miR-430 family. This miRNA family is highly expressed during early zebrafish development, targets hundreds of mRNAs, and is required for embryonic morphogenesis and clearance of maternal mRNAs (A. J. Giraldez et al., Science 308:833-8 (2005); A. J. Giraldez et al., Science 312:75-9 (2006)). It should be noted, however, that the invention described herein is not limited to this particular embodiment, as this embodiment is intended merely as a representative example. Analysis of 3′ UTRs with sites complementary to miR-430 identified squint (sqt), a member of the Nodal family of TGFβ signals, and lft1 and lft2, members of the Lefty family of TGFβ signals (FIG. 5). Nodals are the key regulators of mesendoderm induction and left-right axis formation, whereas Leftys act as antagonists of Nodal signaling (A. F. Schier, Annu Rev Cell Dev Biol 19:589-621 (2003); M. M. Shen, Development 134:1023-34 (2007)). The balance between Nodals and Leftys determines the extent of mesendoderm formation (A. F. Schier, Annu Rev Cell Dev Biol 19:589-621 (2003); M. M. Shen, Development 134:1023-34 (2007); Schier and Talbot, Annu Rev Genet 39:561-613 (2005)) (FIG. 5). Given their potent and concentration-dependent effects, we hypothesized that miR-430 might be required to dampen these signals.

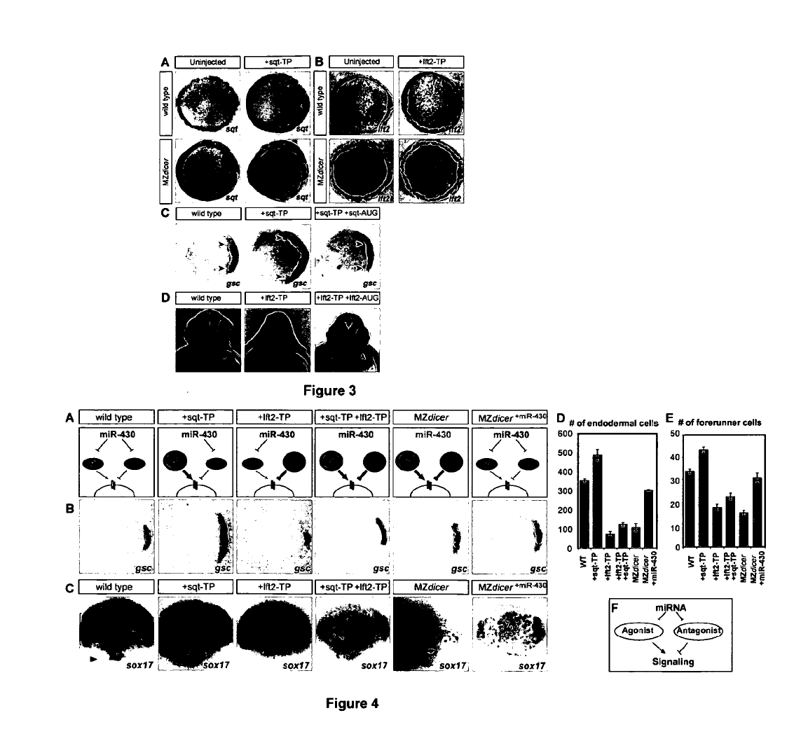

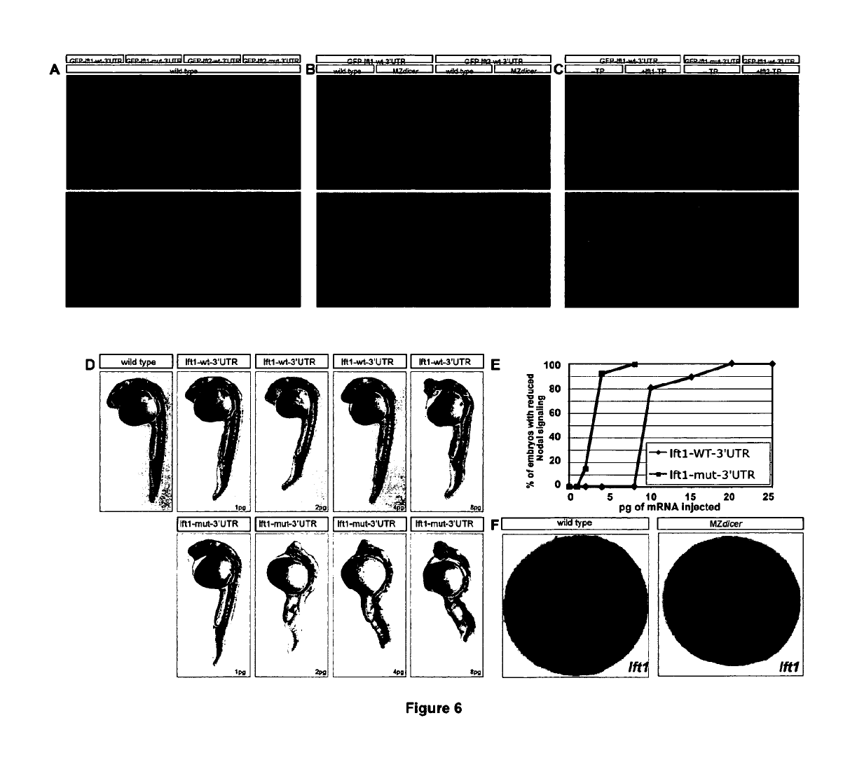

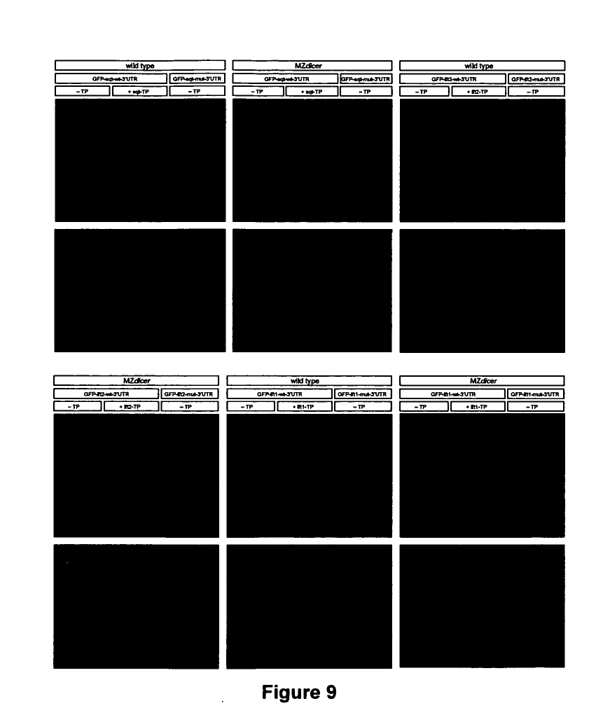

Four lines of evidence indicate that sqt, lft1, and lft2 are in vivo targets of miR-430. First, reporter mRNAs consisting of the GFP coding region and full length sqt, lft1, or lft2 3′UTRs were repressed in wild type but not in MZdicer mutants, which lack all mature miRNAs including miR-430. Derepression of reporter genes was most pronounced for lft2 and least marked for lft1, suggesting that lft2 is more strongly repressed by miR-430 than sqt and lft1 (FIG. 1A, FIG. 6, FIG. 9). Second, mutations of two nucleotides within the miR-430 target site (GCACUU to GGUCUU) abolished repression of reporter mRNAs (FIG. 1A, FIG. 6). Third, endogenous expression of sqt, lft1, and lft2 mRNAs was increased in MZdicer mutants (FIGS. 3A and 3B, FIG. 6). Fourth, misexpression of sqt, lft1, or lft2 mRNAs containing mutated miR-430 binding sites (sqtmut-3′UTR, lft1mut-3′UTR, lft2mut-3′UTR) resulted in higher physiological activity (FIG. 1B to 1F, FIG. 6). These results indicate that miR-430 represses sqt, lft1, and lft2 expression and activity.

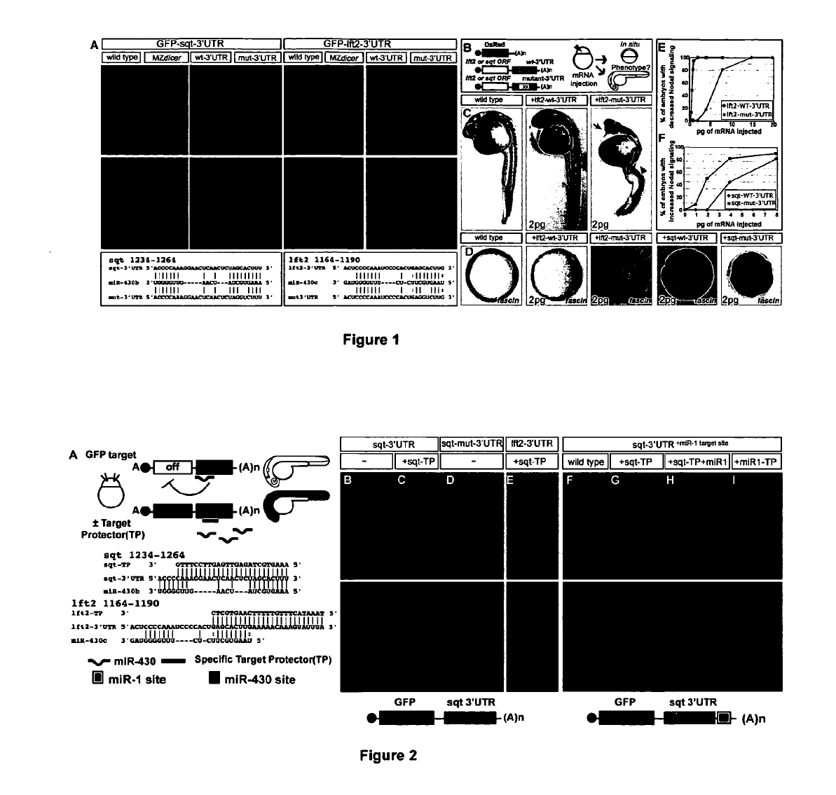

To study the physiological role of miR-430/sqt and miR-430/lft interactions, we developed a method to disrupt the interaction of miRNAs with target mRNAs. RNA-binding morpholino antisense oligonucleotides have been used in zebrafish to block the translation or splicing of target RNAs (J. Summerton, Biochim Biophys Acta 1489:141-58 (1999); Nasevicius and Ekker, Nat Genet 26:216-20 (2000); Draper et al., Genesis 30:154-6 (2001)). We hypothesized that morpholinos overlapping with miRNA target sites might interfere with miRNA-mRNA interactions, thus protecting the target from the miRNA (target protector, TP) (FIG. 2A). Specificity would be attained by the sequences unique to the 3′UTR. Thus, we analyzed the effect of morpholinos complementary to the region of the miR-430 target sites in the sqt or lft 3′UTRs.

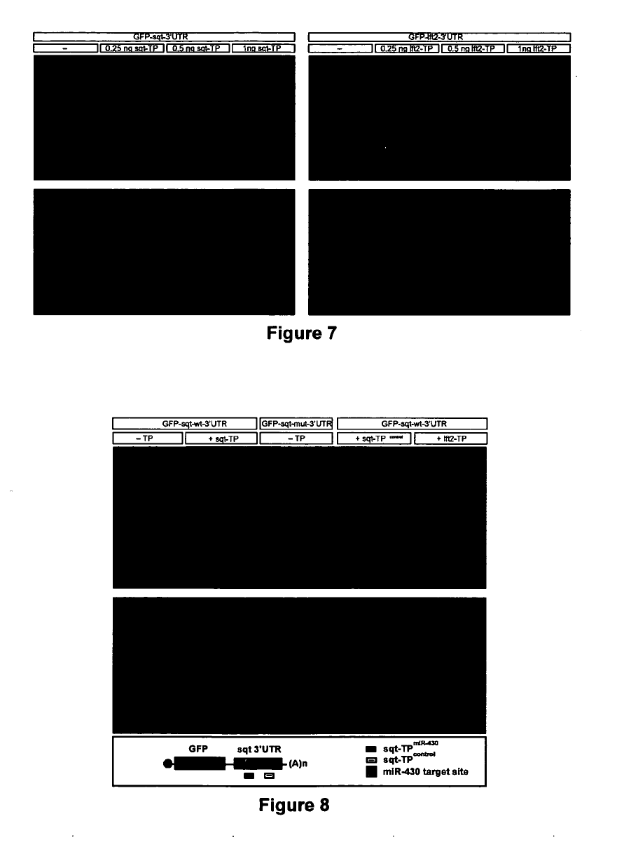

Four lines of evidence indicate that TPs interfere with miR-430-mediated repression of specific 3′UTRs. First, injection of sqt-TP^{miR-430} blocked miR-430-induced repression of the sqt-GFP reporter (FIG. 2B to 2D, FIG. 7). Second, sqt-TP^{miR-430} did not block repression of the lft2-GFP reporter, suggesting that TPs do not induce cross-protection (FIG. 2E). Third, control morpholinos complementary to other regions of the sqt 3′UTR did not prevent sqt-GFP repression by miR-430 (FIG. 8). Fourth, injection of sqt-TPs into MZdicer mutants did not affect the levels of sqt-GFP-reporter or sqt gene expression, suggesting that TPs do not cause non-specific stabilization of mRNAs (FIG. 3A, FIG. 9). Corresponding results were obtained with lft2-TP^{miR-430} (FIG. 3B, FIGS. 6-9).

To test whether TPs specifically block the interaction with one target site without affecting the interaction with other target sites in the same 3′UTR, we placed a miR-1 target site into the sqt-GFP reporter (FIG. 2F to 2I). Protection of the miR-430 target site did not prevent miR-1-mediated GFP repression (FIG. 2H) and protection of the miR-1 target site did not interfere with miR-430-mediated repression (FIG. 2I). Taken together, these results indicate that target protection provides a powerful in vivo method to specifically interfere with and investigate the role of individual miRNA-mRNA target site interactions.

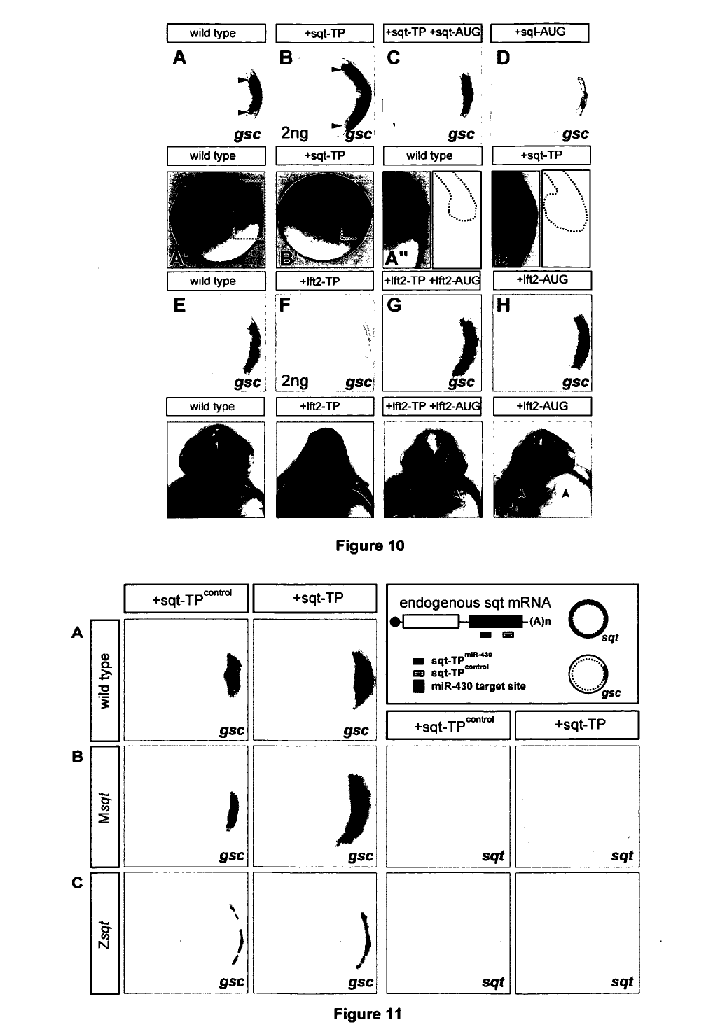

To determine the role of miR-430 repression of sqt, we analyzed sqt-TP^{miR-430}-injected embryos. Similar to MZdicer mutants, sqt expression was elevated (FIG. 3A). Protection of sqt increased the induction of mesodermal marker genes such as goosecoid (gsc), indicative of higher Nodal signaling during blastula stages (A. F. Schier, Annu Rev Cell Dev Biol 19:589-621 (2003); Schier and Talbot, Annu Rev Genet 39:561-613 (2005); B. Feldman et al., Nature 395:181-5 (1998)) (FIG. 3C, FIG. 4B, FIG. 10). The increased gsc induction resulted from the protection of zygotically transcribed but not maternally loaded sqt (FIG. 11). Ectopic gsc induction in sqt-TP^{miR-430}-injected embryos was suppressed by a morpholino blocking sqt translation, indicating that sqt-TP^{miR-430} specifically increased sqt activity (FIG. 3C, FIG. 10). To quantify the effects of increased Nodal signaling, we analyzed the number of sox17-expressing endoderm progenitors and dorsal forerunner cells during gastrulation (A. F. Schier, Annu Rev Cell Dev Biol 19:589-621 (2003); Schier and Talbot, Annu Rev Genet 39:561-613 (2005)). Forerunner cells are induced by Nodal signaling at the dorsal margin and form Kuppfer's vesicle, an embryonic organ that functions during left-right axis formation (Schier and Talbot, Annu Rev Genet 39:561-613 (2005); Essner et al., Development 132:1247-60 (2005)). Cell counting revealed an increase in the number of endodermal and forerunner cells in sqt-TP^{miR-430} embryos (FIGS. 4C, 4D and 4E). These results suggest that miR-430 can dampen Nodal signaling by repressing sqt.

To determine the in vivo role of miR-430 repression of we focused on lft2 because the repression of lft2 by miR-430 was more pronounced than for lft1 (FIG. 1A, FIG. 6). Lft2 target protection resulted in elevated lft2 expression, similar to MZdicer mutants (FIG. 3B). lft2-TP^{miR-430}-injected embryos displayed cyclopia, reduced gsc expression (FIGS. 3D, 4B and FIG. 10) (A. F. Schier, Annu Rev Cell Dev Biol 19:589-621 (2003); Schier and Talbot, Annu Rev Genet 39:561-613 (2005); B. Feldman et al., Nature 395:181-5 (1998)), and fewer sox17-expressing endodermal and forerunner cells (FIGS. 4C, 4D and 4E). These results indicate that miR-430 can enhance Nodal signaling by dampening lft2. To determine the role of miR-430 in simultaneously dampening both sqt and lft2, we coinjected sqt-TP^{miR-430} and lft2-TP^{miR-430}. The induction of gsc was not strongly affected in sqt/lft2-TP^{miR-430} embryos and MZdicer mutants (FIG. 4B), but the expression of sox17 revealed a reduced number of endodermal and dorsal forerunner cells in sqt/lft2-TP^{miR-430} embryos and MZdicer mutants (FIGS. 4C, 4D and 4E). The results indicate that loss of miR-430 regulation leads to an imbalance of sqt and lft inputs and reduces some outputs of Nodal signaling.

In summary, our study of miR-430 and Nodal signaling provides two major insights. First, the regulation of sqt and lft2 by miR-430 identifies a role for miRNAs as dampeners and balancers of agonist/antagonist pairs and reveals a novel regulatory layer of Nodal signaling (FIGS. 4A and 4F, FIG. 5). miR-430 reduces the absolute levels of sqt and lft2 (dampening) and regulates their relative levels to achieve optimal activity of the Nodal pathway (balancing). The protection of sqt and lft2 from miR-430 does not appear to lead to major phenotypic changes during blastula stages (gsc expression) but reduces Nodal signaling during gastrulation (sox17 expression). Since Nodal and Lefty signals have complex regulatory interactions (A. F. Schier, Annu Rev Cell Dev Biol 19:589-621 (2003); M. M. Shen, Development 134:1023-34 (2007)), multiple mechanisms might contribute to this temporal difference. For example, stronger derepression (FIGS. 1A, 3A and 3B) and longer persistence of lft2 after loss of miR-430 regulation could inhibit Sqt and the related Nodal signal Cyclops during gastrulation (A. F. Schier, Annu Rev Cell Dev Biol 19:589-621 (2003); Schier and Talbot, Annu Rev Genet 39:561-613 (2005); B. Feldman et al., Nature 395:181-5 (1998)). The regulation of Nodal signaling by miR-430 is likely to be conserved, because miR-430 is found in other vertebrates (miR-302 (mouse and human), miR-372 (human) and miR-519 (human)) and predicted miR-430 target sites are present in other Nodal and Lefty genes (FIG. 5) (A. J. Giraldez et al., Science 308:833-8 (2005)).

More generally, our results reveal a regulatory interaction in which a repressor (miR-430) dampens the expression of both an agonist (sqt) and an antagonist (lft) (FIGS. 4A and 4F, FIG. 5). Dampening might not only allow balancing of counteracting inputs but also add robustness (Bartel and Chen, Nat Rev Genet 5:396-400 (2004); Hornstein and Shomron, Nat Genet 38 Suppl:S20-4 (2006); Becskei et al., Nat Genet 37:937-44 (2005); Ozbudak et al., Nat Genet 31:69-73 (2002); Raser and O'Shea, Science 309:2010-3 (2005)). For example, our overexpression experiments show that the embryo can tolerate increased levels of miR-430-regulated sqt or lft mRNA, whereas loss of miR-430-mediated regulation leads to gain-of-function phenotypes (FIG. 1C, FIG. 6). miRNA-mediated balancing of agonist/antagonist pairs might also contribute to the evolution of phenotypic changes. The short region of sequence complementarity required for the recognition of miRNA target sites allows for the rapid acquisition, loss, or modulation of miRNA-mRNA target interactions (Chen and Rajewsky, Nat Rev Genet 8:93-103 (2007); Chen and Rajewsky, Nat Genet 38:1452-6 (2006); Clop et al., Nat Genet 38:813-8 (2006)). Our results raise the possibility that target sequence variations could change the balance of agonist/antagonist levels and induce phenotypic changes such as the expansion or reduction of progenitor fields.

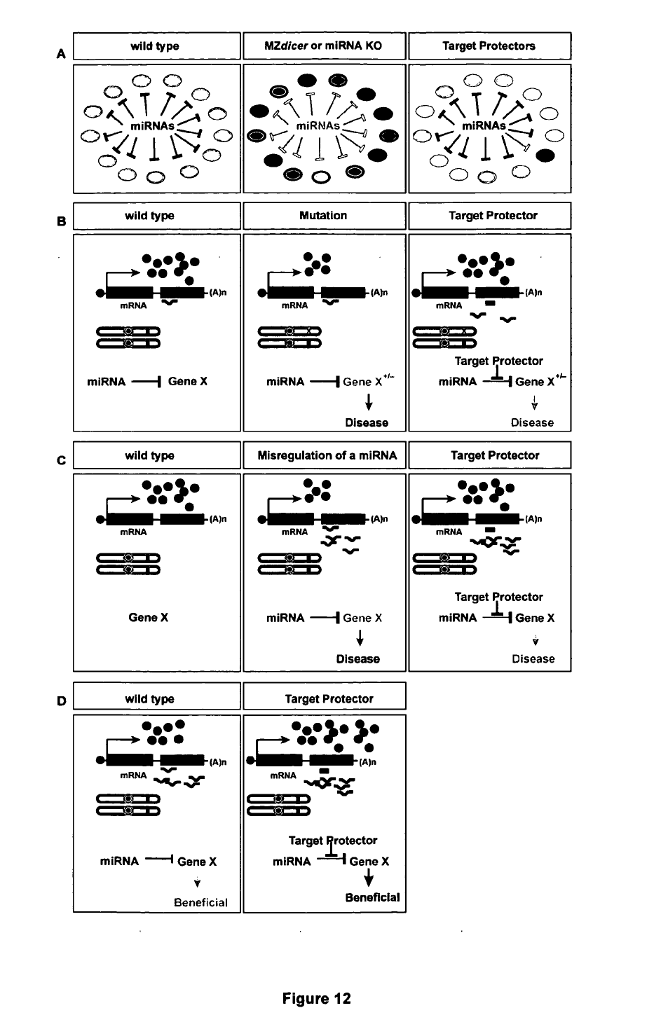

Second, our study introduces a novel method to test the role of specific miRNA-mRNA pairs in vivo (FIG. 12). The sequence selectivity of, e.g., morpholino target protectors makes them excellent agents to disrupt specific miRNA-mRNA interactions. Other antisense oligonucleotides and small molecules that bind to miRNA target sites or their vicinities are also likely to serve as target protectors. Target protectors not only uncover the physiological role of miRNA-mRNA interactions but also illustrate how miRNA phenotypes are a composite created by upregulation of multiple targets (FIG. 12). Additionally, target protectors might serve as therapeutic agents (FIG. 12). More than 30% of all human genes are thought to be miRNA targets (Bushati and Cohen, Annu Rev Cell Dev Biol (2007); Kloosterman and Plasterk, Dev Cell 11:441-50 (2006); Rajewsky, Nat Genet 38 Suppl:S8-13 (2006)). By blocking the interaction of specific miRNA-mRNA pairs using target protectors, the translation and stability of particular mRNAs can be increased and result in the suppression of hypomorphic mutations or the upregulation of beneficial gene products such as tumor suppressors or peptide hormones (FIG. 12).

### Materials and Methods

GFP Reporter and mRNA Misexpression Constructs

GFP reporter: The 3′ UTR of the gene of interest was amplified by PCR from a 0-24 hour post-fertilization (hpf) cDNA library. The primers used for sqt-3′UTR, lft2-3′UTR, and lft1-3′UTR were:

sqt
5′-AAACTCGAGGGCTGCCACTGATTCTTCA-3′,
5′-AAATCTAGACTATTTACAGATAAGGCAAACACG-3′;
lft2
5′-AAACTCGAGAGTGTGGTGTCGAATAGTTTGCT-3′,
5′-CAATCTAGATACTTTATTTTTCAAACATCACATCTC-3′;
lft1
5′-AAAGTCGACGTATAAATAGCCTGTTTGTATCGA-3′,
5′-AAATCTAGAACCCGTGCTATATGCTCA-3′.

PCR fragments were digested and cloned into pCS2+GFP between Xho-Xba.

Mutant GFP reporter: The 3′ UTR of the gene of interest was amplified in two fragments. The two fragments had a ˜40 nt overlap in the mutant region. The mutation in the miR-430 target site was included in the bottom primer of the 5′ fragment and the top primer of the 3′ fragment. The mismatch primers for sqt, lft2, and lft1 were:

sqt
5′-AGGAACTCAACTCTAGGTCTTTGGATATGCTCCTTGACCCC-3′,
5′-GAGCATATCCAAAGACCTAGAGTTGAGTTCCTTTGGGG-3′;
lft2
5′-CCAAATCCCCACTGAGGTCTTGAAAAACAAAGTATTTATGAAT-3′,
5′-ACTTTGTTTTTCAAGACCTCAGTGGGGATTTGGGG-3′;
lft1
5′-TACTAGATACCTCGAGGTCTTTGTATAGACTTGTAAATAGA-3′,
5′-ACAAGTCTATACAAAGACCTCGAGGTATCTAGTAAGGGTT-3′.

The full-length 3′ UTR was obtained by PCR across the mismatch fragments with the 3′UTR primers described above.

sqt/miR-1 GFP reporter: The miR-1 target site was added to the sqt-GFP reporter by ligation of a fragment containing an imperfect miR-1 site:

(5′-ctagctcgagaTACATACTTCTaatCATTCCAta-3′,
5′-ctagtaTGGAATGattAGAAGTATGTAtctcgag-3′).

sqt, lft2 and lft1 full-length mRNA constructs: The gene of interest, including its 3′UTR, was amplified by PCR from a 0-24 hpf cDNA library using the top primers:

sqt
5′-AAACTCGAGCCTTGATTTGACATGTTTTCC-3′
lft2
5′-AAACTCGAGGGCACGAGCATGGCTCTG-3′
lf1
5′-AAACTCGAGCACCTTGAAAAGATGACTTCA-3′.

Bottom primers were the same as for amplifying the 3′UTRs. The mismatches in the miR-430 target site were introduced as described for the GFP reporters.

mRNA Synthesis, Target Validation, miRNA Duplexes and Injection

mrNA for injection was generated using the Message machine kit according to manufacturer's instructions (Ambion). 1 nL of a 0.2 ng/nl GFP reporter mRNA solution was injected into wild-type or MZdicer embryos at the one-cell stage. DsRed control mRNA was co-injected with the GFP reporter mRNA at 0.1 ng/nl.

miRNA duplex: The miRNA duplexes were purchased from IDT and resuspended in the manufacturer's buffer to a concentration of 100 M. Working aliquots were prepared in RNAse-free water at 10 M and stored at −80° C. miR-430 rescue experiments were performed by injecting 2 nl of 10 M miR-430a (Giraldez et al., Science 308:833-8 (2005)). miR-430a, miR-430b and miR-430c share the same seed region. Injection of miR-430a, miR-430b or miR-430c alone has the same ability to rescue MZdicer mutants as a combination of all three miRNAs, suggesting that they are likely to function redundantly (A. J. Giraldez et al., Science 308:833-8 (2005)).

### Misexpression Analysis

sqt misexpression: Embryos were fixed when uninjected wild-type embryos reached 50% epiboly and analyzed for fas or gsc expression by in situ hybridization. “Increased Nodal signaling” was scored as gsc staining covering greater than 50% of the animal pole.

lft misexpression: Embryos were scored at ˜30 hpf for the extent of lft overexpression phenotype. Embryos were considered to have “reduced Nodal signaling” when lacking trunk mesoderm and exhibiting cyclopia. fas in situs were performed on embryos fixed at 50% epiboly. 15-25 embryos were scored for overexpression phenotypes at each concentration.

### Target Protector (TP) Nomenclature

The convention for future naming of target protectors is: [gene name]-TP^{miR-x}. In case of multiple target sites for one miRNA in one 3′ UTR, each target protector for successive target sites should be numbered accordingly. For example, a target protector for the 2nd target site of miR-17 in Myc, would be named, Myc-TP miR-17 (2).

### Target Protector and AUG Morpholino Injections

Target protectors used were:

sqt-TPmiR-430
5′-AAAGTGCTAGAGTTGAGTTCCTTTG-3′
sqt-TPcontrol
5′-TTCTTAAATACATATTTTTGGGGTC-3′
sqt-TPmiR-1
5′-ATGGAATGATTAGAAGTATGTAT-3′
1ft2-TPmiR-430
5′-TAAATACTTTGTTTTTCAAGTGCTC-3′
1ft2-TPcontrol
5′-CCATTTCTAAAGCTTACATTACATA-3′
lft1-TPmiR-430
5′-AAAGTGCTCGAGGTATCTAGTAAGG-3′

Target protectors used in this study were 25-nucleotide long morpholinos designed to bind to the region of the target mRNA complementary to the miRNA seed region (6-8 nt miRNA target site) and to flanking sequences in the 3′UTR. One way to optimize the specificity of TPs is to design the 3′ end of the TP to bind to the target site while the 5′ region of the TP binds to the unique downstream flanking sequences in the 3′UTR. Alternatively, TPs can be designed to have their 5′ end bind to the target site while the 3′ region of the TP binds the upstream flanking sequences in the 3′UTR. Sequence specificity should be confirmed using whole genome sequence alignment programs such as BLAST. All morpholinos and target protector oligonucleotides were obtained from Gene Tools. 1 ng of each target protector was injected at the one-cell stage for target protection experiments. Rescue experiments were performed by injecting 2 ng of sqt-AUG or lft2-AUG morpholinos that block translation.

AUG morpholinos sequences were:

sqt-AUG MO (Feldman and Stempel, Genesis 30: 175-7 (2001))

5′-ATGTCAAATCAAGGTAATAATCCAC-3′

lft2-AUG MO (B. Feldman et al., Curr Biol 12:2129-35 (2002))

5′-AGCTGGATGAACAGAGCCATGCT-3′

### In Situ Hybridization and Cell Counting

Embryos were staged by morphology as described (Kimmel et al., Dev Dyn 2003:253-310 (1995)), correcting for the developmental delay of MZdicer embryos. Probe preparation and in situ hybridization were performed as described (Schier et al., Development 124:327-42 (1997)). Sox17-stained endodermal and forerunner cells were counted from images of deyolked and filleted embryos (Dougan et al., Development 130:1837-51 (2003)).

### Image Acquisition

Embryos were analyzed with Leica MZ16F, Zeiss Axiolmager.Z1 and Zeiss Discovery V12 microscopes and photographed with Optronics MicroFire 2.2 or AxioCam MRc digital cameras. Images were processed with Zeiss AxioVision 4.4, PictureFrame 2.2 and Adobe Photoshop CS software. The levels of the images where adjusted in Photoshop, and identical modifications were applied to all the images in the same experiment.

### Fish Strains

MZdicer embryos were generated as described (A. J. Giraldez et al., Science 308:833-8 (2005); A. J. Giraldez et al., Science 312:75-9 (2006)). MZdicer mutant embryos were dicer^{hu715/hu715}, dicer^{hu896/hu896} or dicer^{hu896/hu715} (Wienholds et al., Nat. Genetics. 35:217-8 (2003)). The sqt mutant embryos carried the sqt^{cz35} allele (B. Feldman et al., Nature 395:181-5 (1998); Heisenberg and Nusslein-Volhard, Dev Biol 184:85-94 (1997).

1. A method of inhibiting the interaction between an mRNA and a small RNA, comprising contacting the mRNA with an mRNA target protector that binds to said mRNA in a sequence specific manner and inhibits interaction between said small RNA and said mRNA.

2. The method of claim 1, wherein the small RNA is a miRNA.

3. The method of claim 1, wherein the small RNA is a siRNA.

4. The method of claim 1, wherein the small RNA is a piRNA.

5. The method of claim 1, wherein the mRNA target protector is an oligonucleotide.

6. The method of claim 5, wherein the oligonucleotide is at least 80%, 90%, 95%, or 100% complementary to said mRNA.

7. The method of claim 5, wherein the mRNA target protector has one or more modifications.

8. The method of claim 7, wherein the modification is 3′ end or 5′ end modifications.

9. The method of claim 1, wherein the mRNA target protector is an antisense oligonucleotide.

10. The method of claim 1, wherein the mRNA target protector is an RNase H-independent oligonucleotide.

11. The method of claim 1, wherein the mRNA target protector is an RNase H-competent oligonucleotide.

12. The method of claim 1, wherein the mRNA target protector is a morpholino.

13. The method of claim 1, wherein the mRNA target protector is a small molecule.

14. The method of claim 1, wherein the mRNA target protector binds to a target region in the 3′-UTR of said mRNA.

15. The method of claim 2, wherein the mRNA target protector binds to a target region of the target mRNA, said target region being complementary to the miRNA seed region.

16. The method of claim 14, wherein the mRNA target protector further binds to a 3′-UTR flanking sequence of the target region.

17. The method of claim 16, wherein one end of the mRNA target protector binds the flanking sequence, and the other end of the mRNA target protector binds the target region.

18. The method of claim 1, wherein the small RNA is endogenous.

19. The method of claim 1, wherein the small RNA is exogenous.

20. The method of claim 1, wherein contacting said mRNA with said mRNA target protector results in an increase in gene expression of a gene encoding said mRNA.

21. The method of claim 20, wherein said gene has a deficient expression level.

22. The method of claim 20, wherein said gene has a normal expression level.

23. The method of claim 1, wherein the mRNA target protector does not substantially inhibit the expression from said mRNA.

24. The method of claim 1, wherein the stability of the mRNA is increased in the presence of the mRNA target protector compared to that in the absence of the mRNA target protector.

25. The method of claim 1, wherein translation from the mRNA is increased in the presence of the mRNA target protector compared to that in the absence of the mRNA target protector.

26. An in vivo method of claim 1, comprising administering an effective amount of said mRNA target protector to an organism expressing the mRNA and the small RNA.

27. The in vivo method of claim 26, wherein the organism is selected from the group comprising of: human, primate, mouse, rat, cow, pig, horse, goat, dog, cat, frog, zebrafish, fly, worm, and plant.

28. The in vivo method of claim 26, wherein the organism is a human patient in need of treatment for a disease characterized by a deficiency in expression from said mRNA.

29. The in vivo method of claim 28, wherein said mRNA target protector is delivered by oral delivery, intravenous delivery, inhalation, percutaneous delivery, vaginal delivery, or rectal delivery.

30. An in vitro method of claim 1, which is carried out in a cell.

31. The in vitro method of claim 30, wherein the cell is from an organism selected from human, primate, mouse, rat, cow, pig, horse, goat, dog, cat, frog, zebrafish, fly, worm, or plant.

32. A method according to claim 30, wherein said cell is cultured outside of a living organism.

33. The method of claim 1, which is carried out in vitro.
